(19) United States
(12) Patent Application Publication
(10) Pub. No.: US 2010/0292602 A1
(43) Pub. Date: Nov. 18, 2010

(54) SEIZURE FORECASTING, MICROSEIZURE PRECURSOR EVENTS, AND RELATED THERAPEUTIC METHODS AND DEVICES

(75) Inventors: Gregory A. Worrell, Rochester, MN (US); Squire M. Stead, Rochester, MN (US)

Correspondence Address: FAEGRE & BENSON LLP;PATENT DOCKETING - INTELLECTUAL PROPERTY 23/24, 2200 WELLS FARGO CENTER, 90 SOUTH SEVENTH STREET, MINNEAPOLIS, MN, 55402-3901, US

(73) Assignee: MAYO FOUNDATION FOR MEDICAL EDUCATION AND RESEARCH, Rochester, MN (US)

(21) Appl. No.: 12/668,345

(22) PCT Filed: Jul. 11, 2008
(86) PCT No.: PCT/US08/69769

§ 371 Date: May 17, 2010

Related U.S. Application Data
(60) Provisional application No. 60/959,076, filed on Jul. 11, 2007.

(51) Int. Cl. A61N 1/36; A61B 5/0476

(52) U.S. Cl. 600/544; 607/45

(57) ABSTRACT

A multiscale recording and stimulation system for identifying and responding to epileptiform activity. The system includes the use of microelectrode arrays (e.g., 10-100 micron electrodes with 100-500 micron spacing) to monitor iEEG activity of submillimeter regions or islands in the epileptogenic zone of brain tissue (e.g., independent microdomains in the range of 100-1000 micron diameter).

### REFERENCE TO RELATED APPLICATION

This application claims the benefit of U.S. Provisional Application Ser. No. 60/959,076, filed Jul. 11, 2007 and entitled Seizure Forecasting, Microseizure Precursor Events, and Related Therapeutic Methods and Devices, which is incorporated herein by reference in its entirety.

### GOVERNMENT RIGHTS

This invention was made with government support under Grant NS047495 awarded by the National Institute of Neurological Disorders and Stroke. The government has certain rights in the invention.

### FIELD OF THE INVENTION

The invention relates generally to focal human epilepsy, localization of the epileptic brain, prediction of seizures, and therapeutic intervention to prevent or abort seizures.

### BACKGROUND OF THE INVENTION

Within a few decades of the 1920's discovery of the alpha rhythm in human EEG, surgeons were using intracranial EEG recordings (iEEG) to guide epilepsy surgery. A remarkably similar approach is still used today, with narrow bandwidth recordings (1-100 Hz) from large, widely spaced electrodes. However, recent research provides strong evidence that the spatial and temporal bandwidth of iEEG currently used to localize epileptogenic brain for epilepsy surgery, and guide responsive brain stimulation to prevent or abort seizures is inadequate.

Epilepsy affects over 50 million people worldwide, and for approximately 30% of the 2.5 million Americans with epilepsy the seizures are not controlled by available therapies. One of the most disabling aspects of seizures is the unpredictability of their occurrence, which severely restricts the lives of people with epilepsy. Partial epilepsy, i.e. seizures that begin in a focal region of the brain, represents the most common type of medically resistant epilepsy. Currently the treatment options are limited to epilepsy surgery, vagus nerve stimulation, or experimental brain stimulation or medications. Currently, epilepsy surgery has the best chance of producing a cure, i.e. complete seizure freedom, but is generally a viable option only if the brain region generating seizures can be localized and safely removed. Unfortunately, many patients are not candidates for epilepsy surgery because the seizures cannot be adequately localized or originate from eloquent cortex that cannot be removed without significant neurological deficits.

Two 1^{st} generation anti-epileptic devices are currently undergoing multicenter trials, the Neuropace Inc. responsive neurostimulator (RNS) and the Medtronic Inc. Intercept device. These devices use fundamentally different stimulation paradigms. The Medtronic device is an open-loop system (similar to deep brain stimulators for tremor) that delivers programmed periodic stimulation to the anterior nucleus of the thalamus. The Neuropace RNS device is a closed-loop responsive system that actually records iEEG and uses automated seizure detection algorithms to trigger stimulation to try and abort the seizure. A distinct advantage of neuroprosthestic devices over epilepsy surgery is that they are non-destructive, reversible, and can target multiple brain regions.

The surgical treatment of refractory partial epilepsy is based on the concept that seizures begin in a discrete region of brain, the seizure onset zone (SOZ), and then propagate to a critical volume of adjacent susceptible tissue, the epileptogenic zone (EZ). To obtain seizure freedom, the SOZ and the surrounding EZ must be resected. Unfortunately, the EZ does not currently have an apriori electrophysiological definition, and is only a concept acknowledging that resection of the SOZ does not always lead to seizure freedom. In some patients a focal lesion can be identified on MRI, e.g. tumor, or a vascular malformation. If the EEG can demonstrate that the SOZ co-localizes with the MRI lesion, then complete resection of the lesion can produce a cure. The rate of seizure free outcomes in these selected patients can approach 80-90%, demonstrating the potential efficacy of epilepsy surgery. However, many patients do not have an MRI lesion, or the lesion extends into functionally eloquent brain, e.g. brain regions supporting language, that cannot be removed without causing a significant neurological deficit. Unfortunately, these patients make up 20-30% of pre-surgical evaluations at major epilepsy centers. For patients with normal MRI scans only 30-50% of those finally deemed surgical candidates will achieve seizure freedom, demonstrating the current limitation of epilepsy surgery.

The gold standard for localizing epileptogenic brain remains iEEG, and patients routinely undergo implantation with subdural and depth electrodes to localize the brain region(s) generating seizures. Unfortunately, without a MRI lesion there is no definition beyond the SOZ for what should be resected.

There has been significant interest in the use of direct brain stimulation for treatment of partial epilepsy. The Neuropace RNS device has been used for patients with well-localized neocortical and hippocampal seizures. The RNS is a closed-loop programmable device that monitors real-time iEEG and delivers electrical stimulation with detection of seizure onset. One embodiment of the system uses subdural strip electrodes composed of four 4 mm diameter contacts spaced 10 mm apart. Preliminary results from the clinical trial have demonstrated that the responder rate, defined as a 50% reduction in total disabling seizures, has ranged from 35% of patients to 41% of patients. Consistently aborting seizures after they are detected on the macroelectrodes has proven difficult.

The predictability of epileptic seizures remains an open problem. A number of iEEG studies have reported quantitative metrics, e.g. signal energy, phase synchrony, correlation dimension, and Lyapanov exponent, to identify a pre-ictal period that precedes the onset of seizures. This remains an active area of research.

Neuronal oscillations that are characteristic of human brain electrophysiology span a wide range of spatial and frequency scales. The spatial organization of neuronal assemblies generating local fields range from small neuronal clusters, to cortical mini-columns and columns (100-600 μm), to large centimeter scale networks. Similarly, the frequencies of oscillation span a wide range from direct-current (DC) fluctuations to very high frequency oscillations (DC-1000 Hz). Research has shown that neuronal oscillations and epileptic energy fluctuations occur as a hierarchy of activity over this wide range of spatiotemporal scales. It has been demonstrated that the magnitude of epileptic energy fluctuations are inversely related to their probability of occurrence, and exhibit long-range temporal correlations with power law scaling. In a related manner, very high frequency oscillations tend to be localized to small networks while slow oscillations are more spatially distributed, recruiting larger networks that modulate the localized fast oscillations.

Studies investigating high-frequency epileptiform oscillations (HFEO) report gamma oscillations (˜40-80 Hz) that are believed to play a fundamental role in feature binding, learning and memory, and ripple oscillations (˜80-200 Hz) that may be important for memory consolidation. Researchers have also reported pathological high-frequency oscillations in human epileptic brain both interictally (between seizures) and at seizure onset. Very high-frequency oscillations (250-700 Hz, coined fast ripples) in mesial temporal lobe epilepsy appear to be localized to the temporal lobe generating seizures, and not present in the contralateral non-seizure generating temporal lobe. Accumulating experimental evidence supports the role of high-frequency epileptiform oscillations, and in particular fast ripples, as an electrophysiological signature of epileptogenic brain.

Pathologic interictal and ictal HFEO (>65 Hz-100 Hz) in human neocortical epilepsy that accurately localized the region of seizure onset has been reported. When compared with the spatial distribution of interictal epileptiform spikes and sharp waves the HFEO were more accurate interictal markers of the region of seizure onset. Additionally, the high-frequency energy in the seizure onset channel was increased, compared to randomly sampled baseline, prior to the onset of seizures in 65% of patients. First generation automated detectors have been developed and perform as well, and with significantly greater reproducibility, as human experts.

Direct current shifts (DC shift) are characterized by a sustained, or very slowly changing iEEG voltage. DC potential shifts have been described in human seizures and in animal models, but are not routinely recorded in human epilepsy. The physiologic effect of extracellularly mediated DC potentials on transmembrane voltage-gated ion channels suggests itself as a potential contributor to seizure generation. The relationship between DC fluctuations and high frequency oscillations during seizure onset has been investigated in one study of 10 patients. Half of the patients had mesial temporal onset seizures, the remaining patients had neocortical onset seizures or a combination of both. High frequency oscillations were observed in the clinically determined seizure onset leads in 70% of the 65 seizures recorded. The seizure onset frequency ranged from 45 to 360 Hz, and in all the patients the high frequency discharge was temporally associated with a scale DC shift ranging from 0.6 to 10 mV. The DC shift preceded the high frequency discharge by seconds in 74% of the seizures, however in other cases the high frequency discharge appeared to precede the DC shift. The DC levels can become unstable in the period preceding seizures and in some cases DC fluctuations may initiate the decline into seizure.

Deep Brain Stimulation (DBS) is an established therapy for Parkinson's Disease, Essential Tremor, Dystonia and emerging therapy for a range of neurological and psychiatric disorders. DBS involves placement of chronic stimulating depth electrodes at specific targets in the brain, and applying duty-cycle stimulation to these brain sites. Success of this therapy depends most significantly upon accurate targeting; the stimulation itself is not sophisticated. Imminently emerging indications for DBS also include Temporal Lobe Epilepsy, Chronic pain, Gilles de la Tourette Syndrome, Major Depression, Obsessive Compulsive Disorder, Lennox Gastaut Syndrome, Minimally Conscious State, and Cluster Headache, each supported by preliminary data. Other proposed indications on theoretical grounds include Spasticity, Dementia, Morbid Obesity, Addiction, and Narcolepsy.

The current delivery system for DBS electrode involves implantation of the deep structures by direct targeting, i.e. passing a rigid depth probe through the superficial cortical structures to the deeper targets. This approach engenders moderate risk of hemorrhage (US average 1%) and infection (US average 3%). Risk of hemorrhage increases with each pass of the electrode, several of which may required during each placement to obtain optimal placement.

DBS surgery is most frequently performed as an awake neurosurgical procedure to allow assessment of the effects and side effects of stimulation at the target site. Placement of the electrodes is most commonly performed with the patient in a stereotaxic head frame to maximize targeting accuracy. This procedure is uncomfortable and requires the patient's head be immobilized for several hours during the surgery.

Cortical stimulation is an established treatment for chronic neuropathic pain, and is widely used as a diagnostic tool to map functional neocortex (language/motor). Other proposed therapeutic indications include dystonia, depression and partial epilepsy. Additionally, recent studies report stimulation mapping for determining the region of epileptogenic brain (Valentin 2002). In these application the modality stimulation are delivered either via epidural or subdural electrodes, and less commonly with depth electrodes that penetrate brain parenchyma.

The therapeutic stimulation paradigms include, but are not limited to chronic duty cycle stimulation, responsive stimulation, and extracellular voltage-clamp (ECVC) stimulation. Diagnostic stimulation paradigms include, but are not limited to wide bandwidth stimulation-response of brain tissue for identifying regions of abnormal brain, such as epileptogenic brain, and continuous or intermittent stimulation-response monitoring for anticipating cerebral dysfunction, such as seizures.

The methods previously proposed to show changes in the scalp or intracranial EEG prior to the onset of seizures have generally used passive recording protocols and analysis. At least one group (Kalitzin, Velis et al. 2005) has used an active stimulation and analysis to identify pre-seizure states prior to seizure onset. However, this approach uses complicated analysis technique that may be difficult to implement in a device.

There remains, therefore, a need for improved methods to spatially and temporally localize epileptogenic brain and seizures to guide resective epilepsy surgery, responsive stimulation with devices, and novel interventional therapies. In particular, there is a need for methods and devices capable of accurately forecasting the onset of a seizures with sufficient lead time to give patients a warning of impending seizures and enable effective interventional therapies that can prevent or mitigate the seizure event.

### SUMMARY OF THE INVENTION

One embodiment of the invention is a multiscale recording and stimulation system or device capable of: 1.) identifying regions of epileptic brain from multiscale recording of spontaneous epileptiform activity and stimulus-induced epileptiform activity, 2.) identifying periods of increased probability of seizures from multiscale recording of stimulus-induced epileptiform activity, 3.) preventing seizures by tailored electrical stimulation delivered in response to microdomain and macrodomain epileptiform activity. Multiscale electrophysiology recording and stimulation approach use arrays of variable size micro and macroelectrodes for recording and electrical stimulation across the range of spatiotemporal scales involved in seizure generation. The microelectrode (e.g., 10-100 micron electrodes with 100-500 micron spacing) arrays are used to continuously monitor the iEEG activity of sub-millimeter regions or islands (e.g., independent microdomains ˜100-1000 micron diameter) throughout the epileptogenic zone of brain tissue and are combined with macroelectrodes (e.g., 1-5 mm diameter with 5-10 mm spacing) that provide large spatial scale information and can deliver electrical stimulation. The macroelectrode electrical stimulation allows direct modulation of the epileptic brain and neuronal populations comprising microdomain activity which is simultaneously monitored by microelectrode recordings. Macroelectrode stimulation is used to: 1.) Control microdomain and macroscale epileptiform activity, including but not limited to aborting microseizures, DC offsets, and high frequency local field oscillations by delivering stimulation that creates a counter field that exactly cancels the ongoing local field activity recorded from multiscale iEEG. Microwire electrode recordings are used to continuously monitor microdomain iEEG, multiunit, and single neuronal unit activity and are used to guide the feedback control signal from the macroelectrodes. 2.) Modulation of epileptic microdomains and measurement of the stimulus-induced epileptiform activity of these regions to identify epileptic brain and states of increased seizure probability, i.e the pre-ictal period. 3.) The generation of focal seizures results from the progressive coalescence of microseizure islands. The microseizures are precursor events to the onset of macroscale seizures, and cannot be detected by macroelectrodes or limited microwire recordings of the type used in the prior art and described above. Identified microseizures can be used alone or in combination with monitored high-frequency epileptiform oscillations (HFEO), DC fluctuations and/or other parameters to identify periods where the brain is in a state of high seizure probability, and guide stimulation or other therapeutic interventions. Additionally, stimulation of regions of epileptic brain and simultaneous microdomain recordings can be used to identify regions of epileptic brain and to identify periods of increased probability of seizure occurrence

### BRIEF DESCRIPTION OF THE DRAWINGS

FIGS. 1A and 1B are a volume rendered MRI brain and an intraoperative photograph showing electrode grids positioned over regions of MRI abnormality (indicated by dashed lines). The clinical subdural grids are composed of an array of 4 mm diameter macroelectrodes separated by 10 mm.

FIG. 2 is an illustration of a 4 mm clinical electrode and the relatively large volume (macrodomain) sampled by the electrode (˜105 neurons) versus a 0.04 mm microwire electrode in accordance with an embodiment of the invention. As shown, the cortex is organized into columns of neuronal clusters, ˜0.03 mm diameter (cortical mini-columns: 100 neurons) to 0.6 mm (cortical columns: ˜7,500 neurons).

FIG. 3 is a graphical illustration of the frequency range of neuronal oscillations and conventional iEEG recordings (0.1-100 Hz).

FIG. 4A is an illustration of EEG signals taken from tissue on adjacent macroelectrodes and microelectrodes. A focal microseizure leading to a clinical seizure is evident on the microelectrode EEGs, but is not seen on the adjacent clinical macroelectrodes. The macroelectrode seizure discharge begins with a DC shift and occurs approximately 20 seconds after the onset of the microseizure event.

FIG. 4B is a histogram of the number of microseizures occurring over 24 hours preceding the first clinical seizure (results from 10 patients). It is evident from this histogram that in the hour before seizures there is a significant increase in the occurrence of microseizure events.

FIG. 5A is a graphic and schematic illustration of a hybrid grid of 24 macroelectrodes and 104 microwire electrodes that can be used in accordance with the invention.

FIG. 5B is a graph of microseizure detections over a two hour period prior to the onset of a clinical macroscale seizure. The upper 24 channels are from the macroelectrodes of the grid shown in FIG. 5A, with seizure onset shown at 7200 seconds. The 104 channels of microseizure detections are shown at the bottom of the graph, and demonstrate the frequent occurrence of microseizure events before the macroscale seizure.

FIG. 5C is an illustration of multiscale iEEG taken with the grid shown in FIG. 5A, with the 24 macroscale electrode signals shown at the top, and the 104 microelectrode signals shown at the bottom. A characteristic microseizure event is highlighted in red in the microelectrode signals.

FIG. 6 is an illustration of a hybrid electrode that can be used to monitor microseizures and/or macroseizures in accordance with the invention. Radial arrays of 40 μm microelectrodes, and macroelectrodes, are shown.

FIG. 7 is an illustration of a portion of the electrode shown in FIG. 6 in tissue and surrounded by microdomains (e.g., about 1000 neurons) at three sequential time periods, illustrating the coalescence of microseizures (red). A microdomain (˜1000s of neurons) and microwire are also shown to scale.

FIG. 8A is a tracing of an automated HFEO detection event at 150 ms. FIG. 8B is a tracing of filtered (>70 Hz) data of the detection event. FIG. 8C is the time-frequency spectrogram of the HFEO event. The event is characterized by an intense spectral peak at about 400 Hz.

FIG. 9 is a histogram showing the total number of detected interictal HFEO events v. frequency (shaded rectangles). The total HFEO count (grey upper line), from microwire microelectrodes (blue middle line) and from macroelectrodes (dashed lower line) are shown and were fitted using a dual mode Gaussian model (N=10). As shown, the average ripple frequency range oscillation for the microwire electrodes (145 Hz) was significantly different compared to that of the macroelectrode (123 Hz). The inset figure shows the number of HFEO detections in a seizure onset zone versus non-epileptogenic brain with microelectrodes and macroelectrodes.

FIG. 10 is a schematic of seizure generation and sites for intervention.

FIG. 11 is an illustration of a multiresolution or multiscale electrode in accordance with one embodiment of the invention located within a patient's vein. The electrode includes microwire array electrodes W (microelectrodes) and macro electrodes M on a body or carrier. The electrodes of the microwire array are generally flush with the carrier.

FIG. 12 is an illustration of another embodiment of a multiresolution or multiscale electrode in accordance with the invention located within a patient's vein. The electrodes of the microwire array extend or protrude from the carrier.

FIG. 13 is an illustration of another embodiment of a multiresolution or multiscale electrode in accordance with the invention located within a patient's vein. The electrodes of the microwire array extend or protrude from the carrier and extend through the vein into adjacent (e.g., brain) tissue.

FIG. 14 is an illustration of a multiresolution or multiscale electrode in accordance with another embodiment of the invention located within a patient's vein. The electrode includes microwire array electrodes W that extend from ports in the carrier.

FIG. 15 is an illustration of a multiresolution or multiscale electrode in accordance with another embodiment of the invention located within a patient's vein. The carrier is tubular to allow blood flow. The carrier can be expandable from a reduced diameter state (not shown) at which the electrode is delivered, to the expanded diameter state shown at which the carrier can engage the inner surface of the vein.

FIG. 16 is an illustration of a multiresolution or multiscale electrode in accordance with another embodiment of the invention located within a patient's vein. The carrier is a thin, arcuate member.

FIG. 17 is an illustration of a multiresolution or multiscale electrode in accordance with another embodiment of the invention located within a patient's vein. The microwire array electrodes and macro electrodes are located on wire struts extending from the carrier.

FIG. 18 is an illustration of a multiresolution or multiscale electrode in accordance with another embodiment of the invention located within a patient's vein. The carrier has a lumen for receiving a guide wire (not shown) to facilitate placement of the electrode. The lumen also allows blood flow. Some of the microwire array electrodes extend from a port on the carrier.

FIG. 19 is a block diagram of a device in accordance with the invention including a power, control, monitored signal processing, stimulation signal generation subsystem connected to electrodes in accordance with the invention.

### DETAILED DESCRIPTION OF THE PREFERRED EMBODIMENTS

A multiscale iEEG approach in accordance with one embodiment of the invention utilizes hybrid electrodes composed of microwire arrays that are combined with clinical macroelectrodes. This approach allows continuous recording of single neurons, small neuronal clusters, microdomains of the scale of cortical columns, as well as large-scale iEEG (macrodomain) activity, and permits stimulation (e.g., responsive, intermittent, or continuous) via standard clinical macroelectrodes. Research on patients undergoing evaluation for epilepsy surgery has produced identified electrographic signatures of epileptogenic brain and precursor events that herald the onset of seizures that are detectable using microelectrode arrays. By probing small spatial scales (˜100-1000 μm) seizure-like events, referred to as microseizures, have been identified on isolated sub-millimeter islands of brain. These microseizures are not detected on conventional macroelectrodes, but are clearly evident on adjacent microwire electrodes. Broadband recording from high spatial density microwire arrays (40 μm wires with submillimeter spacing) show highly localized microseizures, high-frequency epileptiform oscillations (HFEO), and DC fluctuations that can effectively localize the epileptogenic zone and seizures. Prior to the onset of macroscale seizures recorded from clinical intracranial macroelectrodes (the macroscale seizures include clinical and subclinical seizure events), there is an increase in microseizure precursor events. The detection of these microseizure events can improve the efficacy of responsive brain stimulation and epilepsy surgery, and can be used to accurately forecast focal human seizures. For example, the delivery of an auditory tone or other warnings can be delivered to the patients when microseizure activity increases indicating an increased probability of seizure occurrence. Alternatively or in addition to the patient warnings, pre-emptive therapies (e.g., electrical stimulation and/or medications) can be delivered to the patient to mitigate or abort seizures before they fully develop.

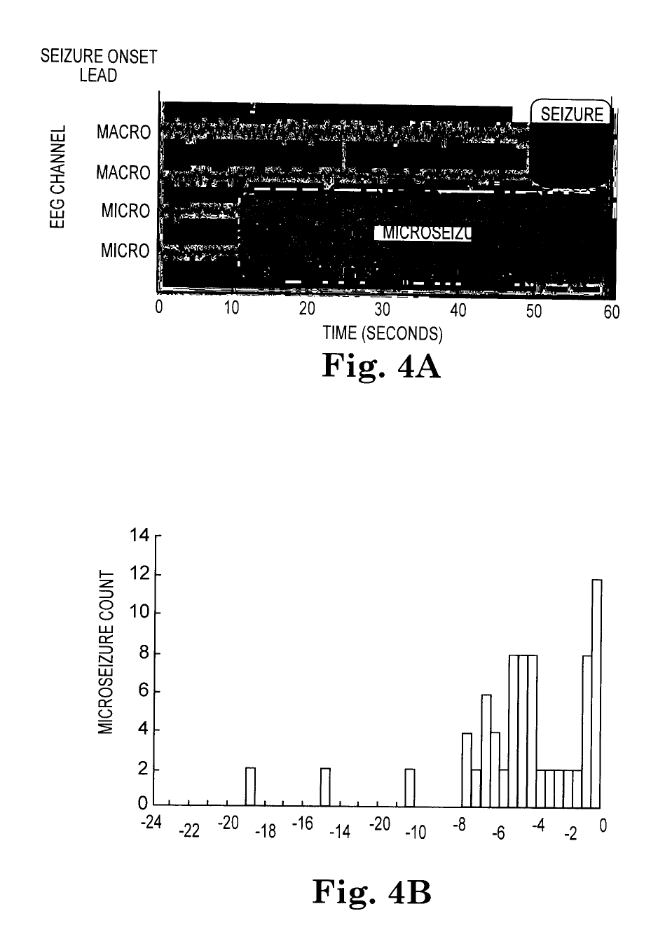

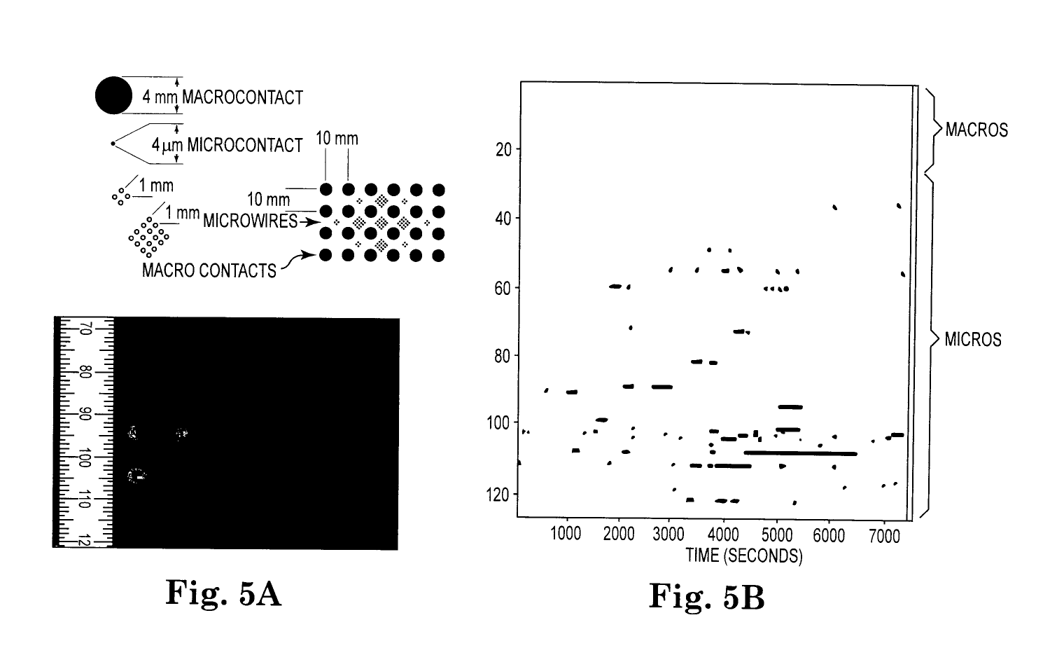

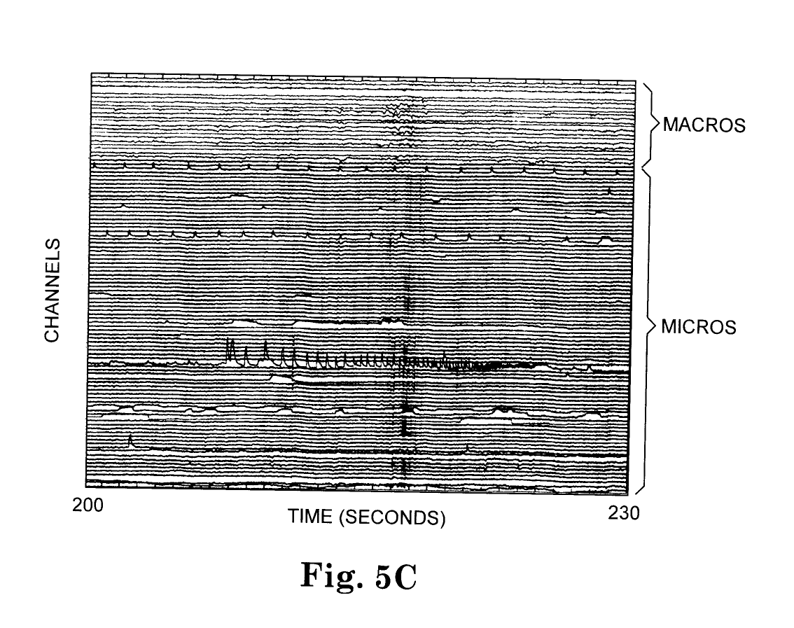

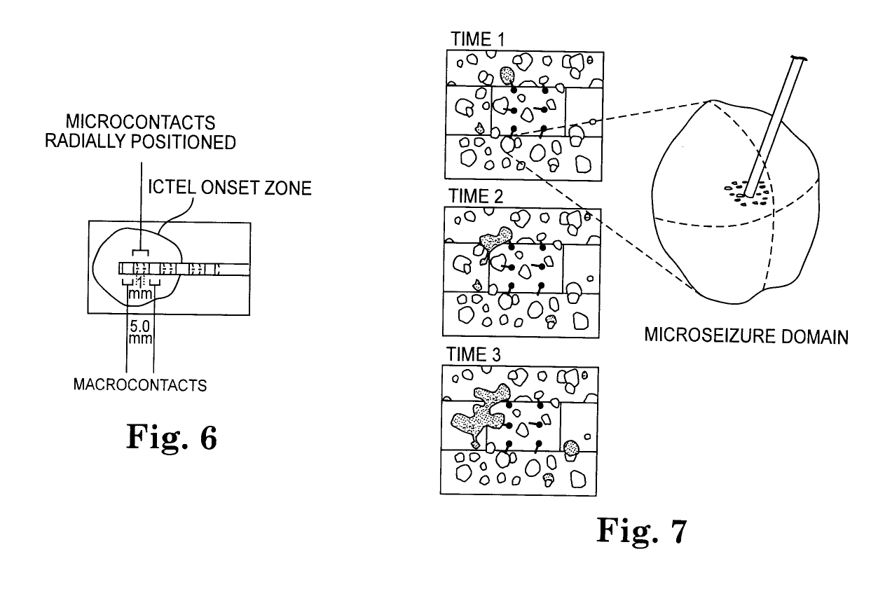

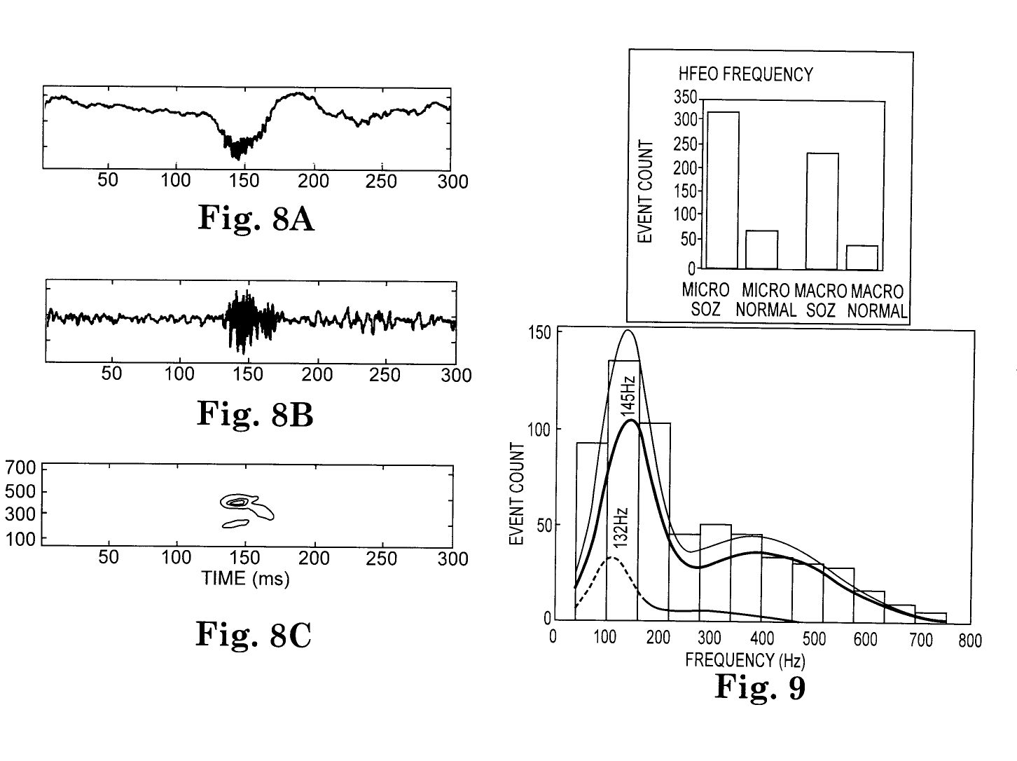

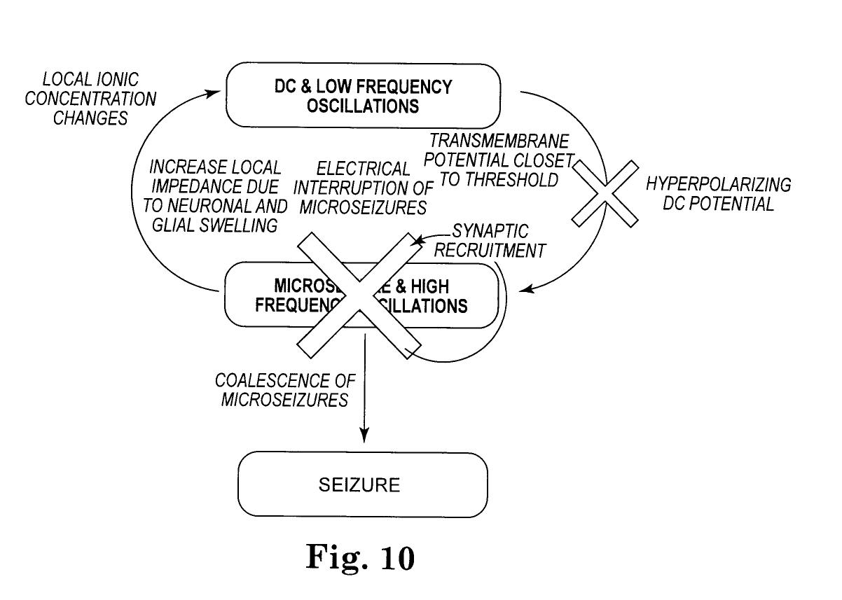

These microscale seizure events are clinically silent, and so spatially localized that they are detectable on the microwire electrodes (e.g., 40 μm in one embodiment) but not on the adjacent macroelectrodes. Microseizures demonstrate spectral characteristics, morphology and durations similar to electrographic seizures detected on macroelectrodes and often are precursors of macroscale seizures. Durations vary between 10 seconds and 10 minutes with a median of ˜30 seconds. They are associated with an abrupt change in the background of the local microscale EEG and evolve in both amplitude and frequency; focal post-ictal spiking and slowing is also frequently observed. In general, the microseizures occur suddenly, and evolve in time and frequency. Less frequently the microseizures demonstrate spatial evolution and recruitment of surrounding seizure that can lead directly to a large scale seizure. Identification of these events can be by visual inspection or through automated detection algorithms. Microseizure data from hybrid depth, subdural strip and grid electrodes demonstrates an increase in the number of microseizures in epileptogenic brain compared to normal cortex and prior to macroelectrode seizures, as shown in FIGS. 4A and 4B. In one embodiment of the invention the iEEG recording was performed using a hybrid grid of 104 microwires embedded among 24 macroelectrodes shown in FIG. 5A.

The application of automated detectors to multiscale iEEG recordings of the type described herein demonstrates that the number of HFEO are significantly localized to the site of the seizure onset zone (SOZ). The microelectrodes show a bimodal distribution of HFEO frequencies with peaks in the ripple (80-250 Hz) and fast ripple (250-800 Hz) range. However, fast ripples were rarely recorded from the clinical macroelectrodes. Consistent with the results obtained using conventional iEEG, it has been determined that neocortical and medial temporal lobe onset seizures are associated with high frequency oscillations. In a study on 10 patients, 70% of seizures demonstrated high frequency oscillations at seizure onset. In the patients with microelectrodes within the seizure onset zone (4 medial temporal lobe patients, 4 neocortical patients), all demonstrated high frequency oscillations in the ripple or fast ripple range as seizure onset.

Multiscale recording and stimulation approaches that probe the relevant spatial and temporal scales involved in the generation of seizures can improve the efficacy of responsive brain stimulation and epilepsy surgery. As is evident from the above discussion, by recording on spatial scales over which the emergence of seizures occur it is possible to identify microseizure precursor events that identify periods of increased probability of seizures and can anticipate the onset of macroscale seizures. Early spatiotemporal localization of seizures can enhance the success of responsive neurostimulation. By the time a seizure is detected using conventional macroelectrodes, a large mass of neural tissue may have been recruited and the seizure can be difficult to abort.

The invention described above can be used: (1) for localization of epileptogenic brain zones, (2) for seizure forecasting and warning, (3) actively probing epileptic brain with electrical stimulation to localize the region of seizure onset and identify periods of increased seizure probability and/or (4) for seizure intervention. These applications can make use of continuous spatiotemporal profiles of microseizure events alone or in combination with high-frequency oscillations and/or DC fluctuations or by detecting the response of microdomain and macrodomain to electrical stimulation. In one embodiment macroelectrode stimulation of epileptogenic brain and detection of microdomain epiletiform activity, such as microseizures, spikes, DC shifts, or HFEO, is used to detect periods of increased probability of seizures. For example, continuous, time dependent probability distributions of the interictal signatures can be developed for: (1) number of occurrence, duration and spatial distribution of the events, (2) a sliding window over multiple temporal scales calculating the measures of the events, and/or (3) updating the probability of occurrence, duration of events and spatial distribution.

During localization of the epileptogenic zone (the EZ region of brain that is removed or lesioned in order to render a patient seizure free), the EZ can be mapped during chronic or intra-operative iEEG recordings to identify microseizures, HFEO and DC fluctuations. Statistical maps of signatures of these events can be co-registered to MRI. Statistically significant regions of increase in the events can then be identified.

Seizure forecasting and prediction can be warning of seizures occurring on a range of time scales (e.g., days, hours or seconds). The invention supports an increase in microseizure activity in the hours (e.g., about 2-6 hours) before clinical or subclinical electrographic seizures. Microseizures also directly progress into macroscale seizures on shorter temporal scales of minutes to seconds. The short-range time scale changes leading to seizure can be associated with: (1) the spread of microseizure activity to adjacent microelectrodes (spatial evolution), (2) increasing synchrony/correlation between microelectrodes EEG, and (3) increasing HFO (e.g., 70-1000 Hz) power present in the microelectrodes and macroelectrodes.

Probabilistic and threshold approaches can be used to determine the threshold level for triggering: (1) seizure warning, (2) intervention and/or (3) localization. A threshold can be determined from base-line non-seizure recording segments (including multiscale base-lines from course grained measures of months, weeks, days, hour and minutes, to fine grained measures of seconds. There can be a continuum of threshold levels that are associated with probability of seizure occurrence. The warning, intervention, or degree of localization can be a threshold at a fixed level or graded.

Based on the probability functions, a warning can be delivered to patients. The warning can be auditory (e.g., a beeper), visual (e.g., a flashing light), a warning to a PDA, broadcast to healthcare providers via a pager, or others.

Interventional stimulation can include electrical stimulation, focal drug delivery and/or focal cooling. Examples of electrical stimulation include real-time extracellular matrix voltage clamp to eliminate microseizures, simple repetitive pulse stimulation, and DC monopolar voltage to hyperpolarize the epileptogenic zone. Example of drugs that can be delivered include benzodiazepines, carbamezapine, lidocaine, ketamine, and others). In the event that a low probability of seizure occurrence is identified, no warning or intervention need be performed.

Extracellular voltage-clamp (ECVC) is a novel approach to altering the behavior of neural tissue by varying an electric field across a region of brain in such a way as to maintain a defined constant (DC) electric potential of the extracellular matrix at the site of a recording electrode, or group of electrodes. Doing so will alter the transmembrane potentials in this regions, moving the neurons further from, or closer to, their action potential firing thresholds. Care should be taken to limit current to acceptable safety margins, although these limits are not likely to be approached with this technique. Records of required correction, mirroring the ongoing, but nulled, field potentials, may be kept digitally as the cumulative sum of the applied voltages for therapeutic or diagnostic analysis. One approach is to apply stimulatory DC energy/field with a device located within a vein of the brain. That is, the stimulatory DC energy/field will be applied to brain tissue from across the venous vessel. Voltage clamping can be used in connection with the activity sensed on the microwire electrodes to vary the amount and/or duration of the voltage clamping.

Diagnostic stimulation protocols are for characterizing brain tissue and the monitoring of brain state and function. Stimulus protocols include, but are not limited to, pulse stimulation response (Valentin 2002), and continuous or intermittent wide bandwidth stimulus response measurements, including the linear response function impedance Z(ƒ). Detection of stimulation response, including impedance, changes will be used for identifying pathologic brain (e.g. epileptogenic brain), changes in brain function and state (e.g. sleep/wake state, disease with paroxysmal cerebral dysfunction such as epilepsy, migraine, cerebral perfusion, stroke, trauma and encephalopathy are associated with impedance change). In particular, the invention includes the application of brain impedance monitoring for identifying time periods of increased probability of seizures in patients with epilepsy. The device can be utilized in patients with epilepsy for warning of impending seizures, or to initiate a therapeutic intervention to prevent seizure occurrence, but is not limited to this application.

The approach applies to wide-bandwidth brain stimulation-response for impedance (linear-response) and higher order response terms measured from scalp, subgaleal, epidural, subdural, intraparenchymal or endovascular electrodes, or combination thereof.

The impedance Z(ƒ) characterizes the linear response to injected time-varying current I(ƒ), i.e. the voltage V(ƒ)=Z(ƒ) I(ƒ), and is the leading term in the Volterra series that describes the response, linear and non-linear response terms, to stimulation. White-noise stimulation currents that contain wide-bandwidth signal can be used to efficiently determine Z(ƒ) across the frequency spectrum and are standard practice in engineering. Alternatively, the response at frequencies of interest can be obtained directly using sinusoidal stimulation.

Any neurological event associated with changes in wide-bandwidth brain impedance could be identified and possibly anticipated with real-time continuous (or intermittent as needed) impedance monitoring. One implementation of this approach would be to seizure prediction, warning and intervention

One embodiment of the invention uses a programmable wide-bandwidth stimulation-response protocol to determine brain impedance (and higher order terms if needed) with variable temporal resolution of impedances (e.g. 1 sec to multiple days). The preferred method achieves several advantages over previous methods reported to potential be useful seizure anticipation:
Low computational complexity and minimal memory requirements
Frequency response, including impedance and higher order terms is easily obtained using current recording and stimulation technology
The algorithm is flexible and parameterized to allow the user to select optimally stimulation parameters (frequency, amplitude).
The approach supports traditional thresholding-based decision making

FIG. 19 is a block diagram of the device in accordance with one embodiment of the invention. The device operates on data from one or more scalp, subgaleal, epidural, subdural, intraparenchymal, endovascular electrodes, or a combination thereof. The device can incorporate multiscale electrodes of the type described herein delivered and placed in the brain by a venous approach. While FIG. 19 shows data from one of each data source, and it is understood that there may be multiple sources of a particular type. It is understood here that “signal metadata” can be included to describe information that is not directly recorded by a sensor. Examples of signal metadata include, sleep/wake state which might originate from a separate sensor, or other device, including a medical device.

The device shown in FIG. 19 can be used to measure the response of a region of a patient's brain using wide-band stimulation. The linear-response (impedance and higher-order response terms from the stimulation are measured. As shown, the device includes a programmable stimulation subsystem for applying an electrical stimulation signal to a region of the brain. The stimulation can include wide bandwidth white noise and sinusoidal stimulation of variable frequency, but is not limited to these modalities. A sensing subsystem detects the response to the electrical stimulation and is coupled to a CPU and analysis module to calculate the frequency or other response signals. An output module initiates interventions or patient reporting. The device components can be enclosed in a biocompatible housing.

Signal filtering can be performed on the data. The device allows for the user to program a range of frequency ranges of interest and the device can use the signal stimulation-response as a feature to best identify changes in brain state and function of interest. The frequency response in the preferred embodiment may include, but is not limited to the following bands (upper/lower cutoff frequencies): (DC/0.1), (0.1/30), (30/80), (80/250), (250/1000), (1000/10,000). It is noted that many suitable filter bands have been identified in the neurological and signal processing literature, and may be employed in addition to, or alternatively to, those listed here.

Signal stimulation response feature extraction is applied to selected outputs. The specific signal stimulation-response features to be applied can be specified by a user interactively or in advance, or default values may be used, and in the preferred embodiment is the brain impedance.

Classification of detected stimulation response events will be programmable to allow a range of classification schemes. Those skilled in the art will recognize that many different classifiers can be used, including kNN, decision trees, neural networks, svms, boosting, linear discriminant analysis. Further, those skilled in the art recognize that classifiers must be trained, and that the details of training, while straightforward, vary by classifier choice.

Cortical electrodes currently utilized for diagnostic and therapeutic stimulation applications include subdural strip and grid electrodes. Multi-resolution or multiscale electrodes of the type described herein (e.g., containing a range of macroscale (˜mm²) and microwire electrodes (˜10⁻³ mm)) can be deployed via the venous vasculature to neocortical sites of interest. Multiple endovascular electrodes can be deployed to approximate a grid array over large regions of neocortex.

Intraparenchymal depth electrodes are currently utilized for stimulation and recording of deep brain nuclei, hippocampus, cortical sulci, and deep cortex (e.g. insula and cingulate cortex). Multi-resolution endovascular electrodes can be implanted via the deep cerebral veins into all the above structures. Again, multiple multiresolution endovascular electrodes can be placed to approximate 1, 2, or even 3 dimensional arrays of electrodes for sensing and stimulating brain tissue.

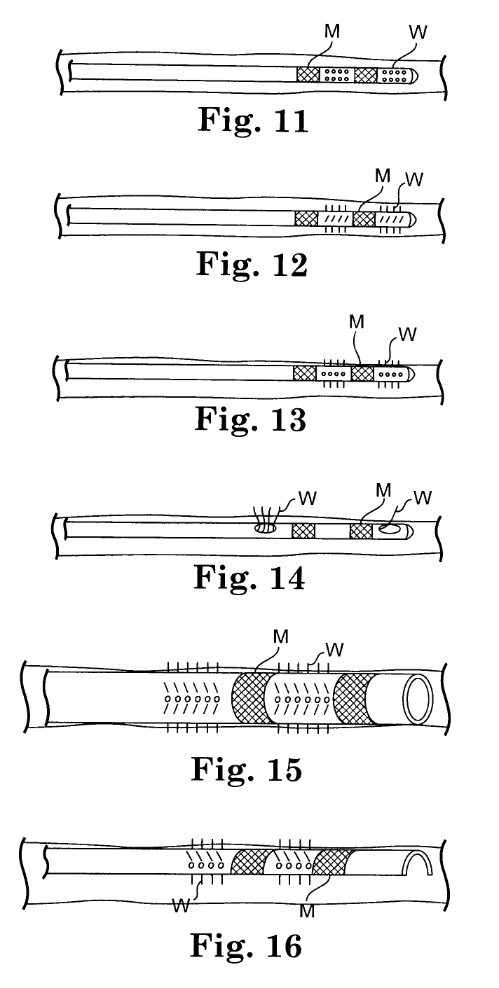

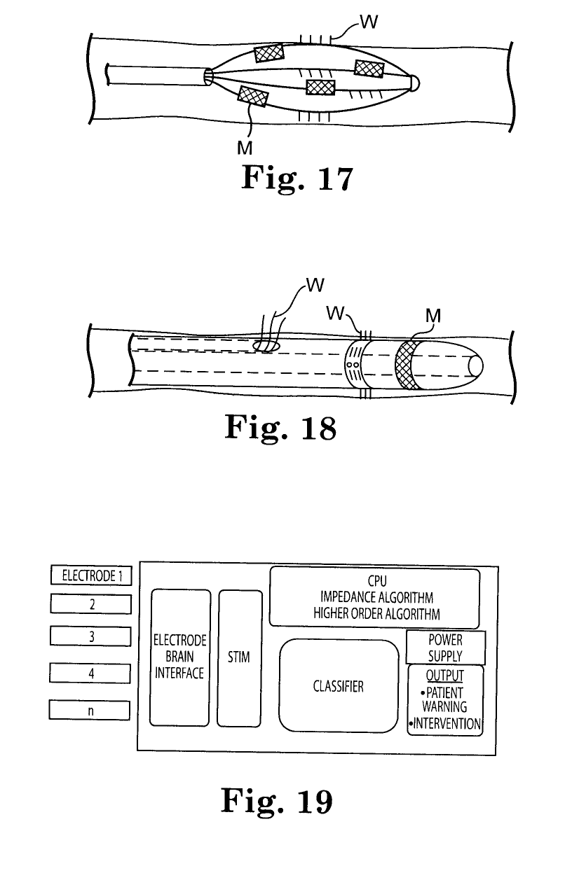

Embodiments of a Multiresolution Endovascular Electrode System (MRES) (i.e., a multiscale electrode) in accordance with the invention are shown in FIGS. 11-18. In FIGS. 11-18 the multiscale electrodes are shown in the venous vasculature which can, for example, be in the patient's brain. Although the illustrated embodiments include two or more microwire electrode arrays W and one or more macro electrodes M on a cylindrical carrier that includes lumens for leads to the electrodes, other embodiments include other combinations of one or more microwire arrays and macro electrodes. The microwire electrode arrays W and macro electrodes M can also be positioned on other structures. As shown in FIG. 15, for example, the MRES can include an expandable hollow cylinder of macroscale or macro electrodes M and microscale or microwire array W electrodes that is deployed within vessels of interest. Upon deployment the expanded hollow cylinder allows blood to flow through the cylindrical electrode. In end vessel venules the MRES may disrupt the vessel wall and occlude the flow of blood.

In some but not all embodiments of the MRES the microwire electrodes penetrate through the vessel wall, and the cylindrical electrode substrate compresses the sight of penetration, thus preventing bleeding.

In yet another embodiment of the MRES the entire electrode is placed outside the vessel wall by allowing the catheter to exit the vessel and penetrating the brain to the site of interest and then deploying the electrode.

With appropriate catheter dimensions, the venous vasculature can be used for access to brain regions within approximately 0.5-1 cm of all potential target sites using established endovascular guidance techniques. The electrode can, for example, be delivered over a previously positioned guide wire. Benefits to the venous approach include the following. The venous anatomy is fixed relative to the brain parenchyma allowing frameless procedures with accuracy equal or better than that afforded by stereotaxic frames. This will allow equivalent success in electrode placement with decreased patient discomfort, and decreased procedure times. Recovery times and hospital stays will be reduced. The prolonged immobility, and subcutaneous tunneling of electrodes required by standard procedures result in significant post-operative pain and recovery delays in many patients. The venous approach will require no period of immobility, and only a short segment of subcutaneous tunneling. Endovascular procedures carry less risk of infection. The majority of the length of the electrode will reside in the vascular system, subject to the patients immune system. Standard DBS electrodes proceed from a sub-galeal location to brain parenchyma, in minimal contact with the blood stream. Similarly, standard subdural and depth electrodes for used in the evaluation of intractable epilepsy are associate with increased risks of infection. Access via the venous system will result in lower risk of hemorrhage. By using the venous approach, the risk of arterial bleeding, present in standard DBS or epilepsy depth electrode procedures is completely averted. The low pressure of the venous system reduces the risk that any hemorrhage induced there will be difficult to control, and thus be of clinical significance.

Potential stimulation sites, therapeutic indications, and venous access points are outlined in the following table:

| Clinical Indication | Target Site | Venous Access Point |
|---|---|---|
| Parkinson's Disease | Subthalamic Nucleus | Basal Vein of Rosenthal |
| | Globus Pallidus Pars | Internal Cerebral Vein |
| | Interna | |
| | Pedunculopontine | |
| | Nucleus | |
| Essential Tremor | Vim Thalamic Nucleus | Internal Cerebral Vein |
| Dystonia | Globus Pallidus Pars | Internal Cerebral Vein |
| | Interna | |
| | Motor/Premotor Cortex | Vein of Rolando |
| Neuropathic Pain | Motor/Premotor Cortex | Vein of Rolando |
| | Periventricular/ | Inferior and Posterior |
| | Periaqueductal Gray | Thalamic Veins |
| | Vc Thalamic Nucleus | Internal Cerebral Vein |
| | Prefrontal Cortex | Vein of Troland |
| | Cingulate Gyrus | Inferior Sagittal Sinus |
| | Insular Cortex | |
| Major Depression | Anterior Cingulate | Inferior Sagittal Sinus |
| | Gyrus, Brodmann Area 25 | Frontal Ascending Veins |
| | Internal Capsule, | Thalamostriate Vein |
| | Anterior Limb | |
| | Nucleus Accumbens | Thalamostriate Vein |
| | Prefrontal Cortex | Vein of Troland |
| Obsessive Compulsive | Internal Capsule, | Thalamostriate Vein |
| Disorder | Anterior Limb | |
| Minimally Conscious | Reticular Activating | Anterior |
| State | System Nuclei of the | Pontomesencephalic |
| | Brainstem | Vein |
| Cluster Headache | Hypothalamus | Basal Vein of Rosenthal |
| Migraine | Hypothalamus | Basal Vein of Rosenthal |
| Morbid Obesity | Hypothalamus | Basal Vein of Rosenthal |
| Narcolepsy | Hypothalamus | Basal Vein of Rosenthal |
| Temporal Lobe Epilepsy | Anterior Thalamic | Internal Cerebral Vein |
| | Nucleus | |
| | Hippocampus | Basal Vein of Rosenthal |
| Neocortical Localization- | Neocortical Seizure | External Cerebral Veins |
| related Epilepsy | Onset Zone | |
| Lennox Gastaut | Centromedian Thalamic | Internal Cerebral Vein |
| Syndrome | Nucleus | |
| Addiction | Nucleus Accumbens | Basal Vein of Rosenthal |
| Gilles de la Tourette | Centromedian Thalamic | Internal Cerebral Vein |
| Syndrome | Nucleus | |
| | Internal Capsule, | Thalamostriate Vein |
| | Anterior Limb | |
| Dementia | Basal Nucleus of | Basal Vein of Rosenthal |
| | Meynert | |
| Spasticity | Motor/Premotor Cortex | Vein of Rolando |
| | Cerebellum | Inferior Vermian Vein |
| Mapping Epileptogenic | Therapeutic Stimulation | Cortical Veins |
| Brain | Mapping Epileptogenic | Basal Vein of Rosenthal |
| Hippocampus | Zone | |
| Neocortex | | |

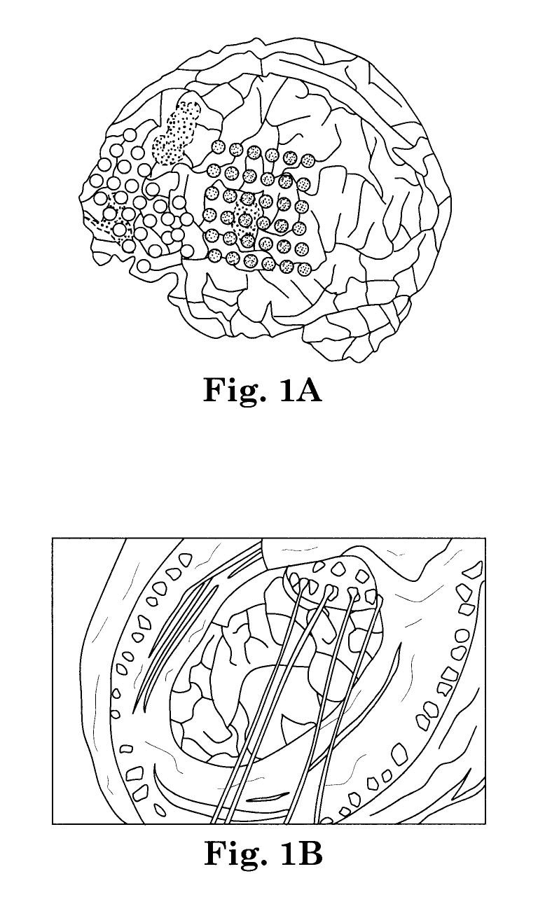

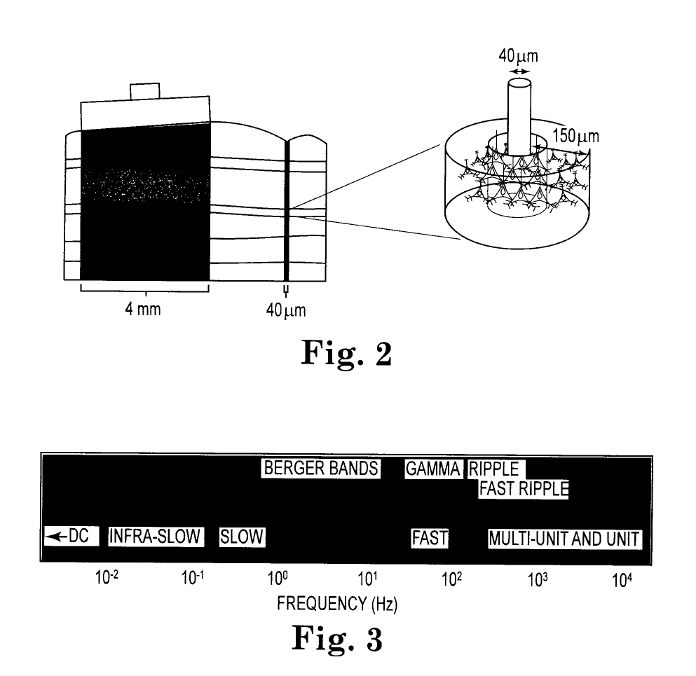

The subsystem of the device such as that shown in FIG. 1 containing the power, logic and control components can be implanted in the subclavian space of the patient. Leads can extend from the subsystem through the patient's venous system (e.g., the jugular vein) to electrodes positioned at one or more zones of interest (e.g., seizure onset zones). The leads can branch into different veins, and one or more electrodes can be located at each of one or more locations. Stimulation and/or sensing electrodes of the types described above can be used with the device. The multiscale electrodes can be removed from the patient following procedures, or permanently placed in regions of the brain previously mapped and known to be a seizure onset zone. Brain activity such as microseizures can be sensed with the microwire array electrodes while therapeutic stimulation (e.g., traditional method, DC or voltage clamp) can be applied through the macro electrodes on the same or a different catheter carrier.

Although the present invention has been described with reference to preferred embodiments, those skilled in the art will recognize that changes can be made in form and detail without departing from the spirit and scope of the invention. In particular, the methods described herein can be implemented by wide variety of devices and systems, and can be used in connection with a wide variety of interventional therapies, including those described in the references below. The subject matter of the following references is hereby incorporated by reference:
1. Berger, H., On the Electroencephalogram of Man. Tenth Report, in Hans Berger on the Electroencephalogram of Man, P. Gloor, Editor. 1935, Elsevier: Amsterdam.
2. Penfield, W., Erickson, T. C., Epilepsy and cerebral localization. 1941, Springfield, Ill.: Charles C. Thomas.
3. Engel, J. J. and T. A. Pedley, Epilepsy: a comprehensive textbook. 1997, Lippincot: Philadelphia, Pa.:
4. Theodore, W. H. and R. S. Fisher, Brain stimulation for epilepsy. Lancet Neurol, 2004. 3(2): 111-8.
5. Worrell, G., et al., Safety and evidence for efficacy of an implantable responsive neurostimulator Epilepsia, 2005. 46: 226 (Abstract).
6. Litt, B., et al., Epileptic seizures may begin hours in advance of clinical onset: a report of five patients. Neuron, 2001. 30(1): 51-64.
7. Mormann, F., et al., Seizure prediction: the long and winding road. Brain, 2007. 130(Pt 2): 314-33.
8. Worrell, G. A., et al., High-frequency oscillations and seizure generation. Brain, 2004. 127: 1496-506.
9. Dzhala, V. I. and K. J. Staley, Transition from interictal to ictal activity in limbic networks in vitro. J Neurosci, 2003. 23(21): 7873-80.
10. Buzsaki, G. and A. Draguhn, Neuronal oscillations in cortical networks. Science, 2004. 304: 1926-9.
11. Vanhatalo, S., J. Voipio, and K. Kaila, Full-band EEG (FbEEG): an emerging standard in electroencephalography. Clin Neurophysiol, 2005. 116(1): 1-8.
12. Bragin, A., et al., High-frequency oscillations in human brain. Hippocampus, 1999. 9(2): 137-42.
13. Worrell, G., et al, Evidence for self-organized criticality in hippocampus. Neuroreport, 2002.13: 2017-21.
14. Parish, L. M., et al., Long-range temporal correlations in epileptogenic and non-epileptogenic human hippocampus. Neuroscience, 2004. 125(4):1069-76.
15. Staba, R. J., et al., Quantitative analysis of high-frequency oscillations (80-500 Hz) recorded in human epileptic hippocampus and entorhinal cortex. J Neurophysiol, 2002. 88(4):1743-52.
16. Bak, P. and M. Paczuski, Complexity, contingency, and criticality. Proc Natl Acad Sci, 1995. 92:6689-96.
17. Buzsaki, G., et al., High-Frequency Network Oscillation in Hippocampus. Science, 1992. 256: 1025-27.
18. Timofeev, I. and M. Steriade, Neocortical seizures: initiation, development and cessation. Neuroscience, 2004. 123(2): 299-336.
19. Traub, R. D., et al., A possible role for gap junctions in generation of very fast EEG oscillations preceding the onset of, and perhaps initiating, seizures. Epilepsia, 2001. 42(2): 153-70.
20. Gardner, A., et al., Human and Automated Detection of High-Frequency Oscillation in Clinical Intracranial EEG Recordings. Journal of Clinical Neurophysiology, 2007. 118: 1134-43.
21. Ulbert, I. et. al., Multiple microelectrode-recording system for human intracortical applications. Journal of Neuroscience Methods, 2001. 106:69-79.
22. Bedard, C., H. Kroger, et al. (2004). “Modeling extracellular field potentials and the frequency-filtering properties of extracellular space.” Biophys J 86(3): 1829-42.
23. Bedard, C., H. Kroger, et al. (2006). “Model of low-pass filtering of local field potentials in brain tissue.” Phys Rev E Stat Nonlin Soft Matter Phys 73(5 Pt 1): 051911.
24. Chebabo, S. R., M. A. Hester, et al. (1995). “Interstitial space, electrical resistance and ion concentrations during hypotonia of rat hippocampal slices.” J Physiol 487 (Pt 3): 685-97.
25. Kalitzin, S., D. Velis, et al. (2005). “Electrical brain-stimulation paradigm for estimating the seizure onset site and the time to ictal transition in temporal lobe epilepsy.” Clin Neurophysiol 116(3): 718-28.
26. Litt, B. and J. Echauz (2002). “Prediction of epileptic seizures.” Lancet Neurol 1(1): 22-30.
27. Litt, B., R. Esteller, et al. (2001). “Epileptic seizures may begin hours in advance of clinical onset: a report of five patients.” Neuron 30(1): 51-64.
28. Litt, B. and K. Lehnertz (2002). “Seizure prediction and the preseizure period.” Curr Opin Neurol 15(2): 173-7.
29. Logothetis, N. K., C. Kayser, et al. (2007). “In vivo measurement of cortical impedance spectrum in monkeys: implications for signal propagation.” Neuron 55(5): 809-23.
30. Mormann, F., R. G. Andrzejak, et al. (2007). “Seizure prediction: the long and winding road.” Brain 130(Pt 2): 314-33.
31. Olsson, T., M. Broberg, et al. (2006). “Cell swelling, seizures and spreading depression: an impedance study.” Neuroscience 140(2): 505-15.
32. Ranck, J. B., Jr. (1970). “Electrical impedance changes in many sites of brain in paradoxical sleep, anesthesia, and activity.” Exp Neurol 27(3): 454-75.
33. Valentin, A (2002) “Response to single pulse electrical stimulation identify epileptogenesis in human brain in vivo” Brain 125:1709-1718
34. Fischell et al. U.S. Pat. No. 6,353,754.
35. Echauz et al. U.S. Pat. No. 6,678,548.
36. Skinner U.S. Pat. No. 7,076,288.
37. Pless U.S. Pat. No. 7,174,213.
38. Wallace et al. U.S. Patent Publication 2005/0137647
39. Litt et al. U.S. Patent Publication 2008/0027346

What is claimed is:

1. A therapeutic method including monitoring microseizures within neural microdomains in the brain.

2. The therapeutic method of claim 1 wherein the microdomains are sub-millimeter islands of brain.

3. The therapeutic method of claim 2 wherein the microdomains are on the order of 1000 μm or less in size.

4. The therapeutic method of claim 1 including monitoring a plurality of the microdomains.

5. The therapeutic method of claim 1 wherein monitoring microseizures includes monitoring iEEG through microelectrodes.

6. The therapeutic method of claim 5 wherein the microelectrodes are on the order of 200 μm or less in size.

7. The therapeutic method of claim 1 wherein the microelectrodes are spaced from one another on the order of 1 mm or less.

8. The therapeutic method of claim 1 and further including performing interventions as a function of the monitored microseizures.

9. The therapeutic method of claim 8 wherein the interventions include one or more of drug delivery, stimulation, and warning.

10. The therapeutic method of claim 8 and further including monitoring macrodomain iEEG and performing interventions as a function of the macrodomain iEEG and the monitored microseizures.

11. The therapeutic method of claim 10 wherein the macrodomain iEEG includes one or both of high-frequency epileptoform oscillations and DC fluctuations.

12. A multiscale electrode array for use with the therapeutic method of claim 10.

13. A passive recording and stimulation-response multiscale electrophysiology method, including monitoring microseizures in microdomain structures, for identifying epileptic brain, periods of increased seizure probability, and therapeutic intelligent control stimulation to prevent and/or abort seizures.

14. The method of claim 13 wherein the monitoring is performed through venous access.

15. The method of claim 14 wherein the simulation is performed through venous access.

16. The method of claim 13 including monitoring the electrical stimulation-induced response and epileptiform activity from a plurality of the microdomains and macrodomain structures to identify epileptogenic brain and probability of seizure occurrence.

17. The method of claim 14 and further including performing interventions including electrical stimulation as a function of the monitored microseizures, DC-shifts or HFEO, and including intelligent stimulation that is tuned to effectively cancel the local field activity over selected frequency bands or the entire spectrum (DC-1000 Hz)
